(19) United States
(12) Patent Application Publication
(10) Pub. No.: US 2009/0041619 A1
(43) Pub. Date: Feb. 12, 2009

(54) DEVICES FOR SANITIZING MEDICAL FITTINGS

(75) Inventors: Timothy B. CADY, Encinitas, CA (US); Mark R. Nowakowski, San Diego, CA (US)

Correspondence Address: Biotechnology Law Group;c/o Portfolioip, P.O. Box 52050, Minneapolis, MN, 55402, US

(73) Assignee: Zinc Medical, Inc., San Diego, CA (US)

(21) Appl. No.: 12/143,787

(22) Filed: Jun. 21, 2008

Related U.S. Application Data
(60) Provisional application No. 60/945,696, filed on Jun. 22, 2007.
(60) Provisional application No. 60/979,819, filed on Oct. 13, 2007.

(51) Int. Cl. A61L 2/18

(52) U.S. Cl. 422/28; 422/292

(57) ABSTRACT

Single-use devices for sanitizing accessible surfaces of needleless medical valves at risk of contamination with infectious agents are described, as are methods for making and using such devices.

### RELATED APPLICATIONS

This application claims the benefit of and priority to U.S. provisional patent application Ser. Nos. 60/945,696, filed 22 Jun. 2007, and 60/979,819, filed 13 Oct. 2007, each of which is commonly owned with the instant application and is herein incorporated by reference in its entirety for any and all purposes.

### TECHNICAL FIELD

This invention concerns small disposable, single-purpose devices useful for sanitizing needleless valves on medical fittings, particularly those surfaces of such valves that are or may be at risk of contamination with infectious agents.

### BACKGROUND OF THE INVENTION

1. Introduction.

The following description includes information that may be useful in understanding the present invention. It is not an admission that any such information is prior art, or relevant, to the presently claimed inventions, or that any publication specifically or implicitly referenced is prior art.

2. Background.

Exposure to infectious agents (e.g., pathogenic bacteria, viruses, fungi, etc.) in medical settings is a matter of serious concern. One route of exposure to such agents is the opening made in skin provided by the bore of needle, canula, or other similar device used to provide access to a patient's vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC). All told, in the U.S. approximately 20,000 patients die annually from hospital acquired infections that result from PIVC and CVC use. Costs associated with the care and treatment of patients that develop infections due to PIVC and CVC use exceed $2.7 billion.

In hospital settings today, occupational health and safety regulations designed reduce the risk to health care workers from needle prick and similar injuries have resulted in the deployment of needleless medical valves whenever possible. Currently, more than 500 million needleless valves are used annually in hospitals throughout the U.S. Needleless valves are used primarily in conjunction with PIVC and CVC devices, which may contain from as few as one to as many as 3, 4, 5, or more needleless valves. FIG. 1 illustrates an example of a representative medical valve in use today.

The widespread use of needleless valves in acute medicine has contributed to a marked increase in the incidence of hospital acquired infections (HAIs), particularly blood stream infections. To reduce the risk of infection from a contaminated needleless valve, standard practice today requires that a nurse or other health care worker clean the surface of a needleless valve by rubbing it with a sterile alcohol swab or wipe immediately prior to making a connection to the valve, for example, attaching a syringe to the valve to deliver a medication via a PIVC already connected to a patient.

Other approaches have also been suggested, such as placing caps on each needleless valve when not being accessed. Examples of such devices include cylinder caps that can be threaded and sealed onto a needleless valve to minimize exposure of the syringe-engaging portion of the valve to air when the valve is not being accessed. The cap houses a sponge element positioned in the threaded cap to contact with the valve surface when the cap is affixed to a needleless valve. As the cap is threaded onto the valve, a crushable reservoir filled with an antiseptic solution is ruptured. Over time, the antiseptic solution flows into the sponge and ultimately reaches the surface of the needleless valve. While such caps may provide an antiseptic environment for an indefinite period, they could not be used to quickly sanitize an uncapped needleless valve, as any such cap would first have to be screwed into place in order to rupture the antiseptic-containing reservoir. The antiseptic would then have to migrate through the already compressed sponge to reach the surface of the valve to be disinfected. Moreover, the use of such antiseptic barrier caps would require that they be deployed at all times on any and all exposed needleless valves in a PIVC, CVC, or other medical line connected to a patient, except when the particular valve is being used. Of course, after a particular valve is used, it would then have to be recapped with a new cap. Such an approach would be expensive and time-consuming, if not impractical.

Other suggested cleaning and capping examples are elastic pouches. When not in use, such pouches have a flat configuration, which can be elastically expanded by squeezing a pouch to form a cavity adapted to receive a needleless medical valve. While such pouches lack threads, they, like the threaded caps mentioned above, are designed to be left in place on a needleless medical valve, and also serve as caps, some of which may also include elements other than threads to provide relatively secure attachment to a needleless medical valve.

Simply put, existing approaches leave much to be desired, as evidenced by the large number of blood stream infections that result from PIVC and CVC use. Clearly there is long-recognized yet unmet need for devices that can be quickly and easily used to sanitize needleless medical valves.

3. Definitions.

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “aqueous solution” refers to a water-based solution capable of dissolving or dispersing one or more other substances, or solutes (i.e., the substance(s) dissolved in the solvent). A “solution” is a homogeneous mixture of at least one substance in a liquid. In the context of this invention, “aqueous solvents” can also include other liquids, including organic liquids, such as alcohols and/or oils.

An “infectious agent” refers to any organism capable of infecting another organism. Such agents include many bacteria, viruses, and fungi.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

In a “suspension” solid particles are dispersed in a liquid. The term “colloidal” refers to a state of subdivision, which, in the context of solutions, means that molecules or particles dispersed in the liquid have at least in one direction a dimension roughly between 1 nm and 1 μm. It is not necessary for all three dimensions to be in the colloidal range. A “colloidal dispersion” is a system in which particles of colloidal size of any nature (e.g. solid, liquid or gas) are dispersed in a continuous phase of a different composition (or state). In an “emulsion” liquid droplets and/or liquid crystals are dispersed in another liquid. An emulsion may be denoted by the symbol “O/W” if the continuous phase (i.e., is an aqueous solution) and by “W/O” if the continuous phase is an organic liquid.

### SUMMARY OF THE INVENTION

It is an object of this invention to provide patentable single-use sanitizing devices that can be used to effectively and efficiently sanitize, and preferably sterilize, exposed surfaces of needleless medical valves, particularly the accessible surface of the valve stems of needleless valves of medical fittings, particularly those surfaces that may become contaminated with infectious agents. In the context of the invention, “sanitize” encompasses cleaning, disinfecting, and/or sterilizing.

Sanitizing devices, or articles, according to the invention are preferably pre-packaged, sterilized single-use devices that, once used, can be disposed of. They are not structurally configured, nor are they intended, to serve as caps or other semi-permanent covers for the surface(s) of needleless medical valve; instead, each is designed to be used to sanitize an exposed needleless medical valve, after which the used sanitizing device is immediately disposed of. In some embodiments, the devices are used manually, whereas in others, one or more of the devices are inserted (individually or in magazines) into a hand-held machine that, when properly positioned in relation to a needleless medical valve, allows the exposed surfaces of the valve to be sanitized upon actuation of the machine.

Thus, one aspect of the invention concerns patentable single-use sanitizing articles configured to sanitize needleless valves of medical fittings. Such articles typically comprise a sanitizing element integrated with a shell or housing. A sanitizing element comprises a substrate and a sanitizing reagent dispersed in the substrate prior to use, preferably at the time the device is manufactured. In some embodiments, however, the sanitizing reagent may be released for dispersion into the substrate post-manufacture, but prior to the time the device is brought into contact with the needleless valve to be sanitized. The sanitizing element substrate includes a sanitizing region capable of engaging an accessible surface of a valve stem of a needleless medical valve so as to expose the accessible surface, and any infectious agents residing thereon, to the sanitizing reagent. In some embodiments, the sanitizing element comprises a single layer, whereas in others, it comprises a plurality of layers. In multi-layer devices, the substrate used to form each layer may be of the same or different material, and may or may not contain a sanitizing reagent. When two or more layers each contain a sanitizing reagent, it may the same or different. Additionally, in some embodiments of multi-layer devices, one or more of the layers may be physically separated from the other layer(s) by an impermeable, semi-permeable, or permeable barrier.

In preferred embodiments, the substrate used to form the sanitizing element is any suitable absorbent, pliable fibrous or porous material, or combination of materials that can be wetted and/or impregnated with a sanitizing reagent. Such materials include those that are synthetic or naturally occurring, and they may be of homogeneous or heterogeneous composition. Preferred synthetic materials include fibrous, foam, and gel compositions. Preferred natural materials include those derived from cotton and naturally occurring sponges. With respect to synthetic fibrous materials, those having directly oriented fibers are particularly preferred. In embodiments wherein the sanitizing element is comprised of two or more layers, the substrate portion of each layer can be formed from a material that is the same as or different from the material used to form the substrate of one or more of the other layers, and each layer may contain the same, different, or even no, sanitizing reagent (although at least one layer will have a sanitizing reagent dispersed therein prior to engaging the surface of the needleless valve to be sanitized). Also, even when substrates for different layers are formed from the same material, they may be configured differently. For example, in a particularly preferred embodiment that employs a sanitizing element having two layers, where the substrate for each layer is formed from the same type of synthetic absorbent material having directionally fibers, the orientation of the fibers in one layer can differ from the fiber orientation in the other layer.

In still other embodiments having multi-layer sanitizing elements, the sanitizing region comprises a material having an abrasive or scrubbing quality that differs from the other layer(s), in order to achieve improved sanitizing of the potentially contaminated exposed surface(s) of a needleless medical fitting valve. Such an abrasive layer may or may not comprise a sanitizing reagent dispersed therein during manufacture; however, any such layer allows sanitizing reagents disposed in other layers of the sanitizing element to reach the valve surface(s) to be sanitized during a sanitizing procedure.

In the articles of the invention, the sanitizing element is disposed in any suitable shell. In many embodiments, the shell is a prefabricated. Suitable shells include those formed from cast, extruded, molded, or vacuum-formed materials, particularly plastics of suitable quality and relative impermeability for use in the context of the invention. Other suitable shells include those made from foils and similar materials into which a sanitizing element according to the invention can be inserted or formed. In still other embodiments, a shell can be formed as a coating applied to a sanitizing element.

In general, the single-use sanitizing articles of the invention are provided to users in a sealed, sterile manner. Typically this involves securing a seal to the article to cover the sanitizing region of the substrate, thereby enclosing the sanitizing element, particularly when the shell is comprised of a preformed solid material (e.g., plastic). After sealing, such sanitizing articles are preferably packaged into a suitable container, for example, a foil pouch, for storage and transport. In embodiments that comprise a material that itself is sealable, for example, a foil, an additional seal to cover the sanitizing element is not typically included in the device. In particularly preferred embodiments, packaged sanitizing articles are sterilized using a suitable process, such as irradiation. As will be appreciated, sanitizing articles may be packaged individually or in groups of two or more units as kits, which can further include instructions for use of the sanitizing article(s).

Other aspects of the invention relate to methods of making and using the single-use sanitizing articles of the invention, as well as to methods for reducing a patient's infection risk. Still other aspects concern hand-held machines that use sanitizing articles of the invention to sanitize needleless medical valves.

Other features and advantages of the invention will be apparent from the following drawings, detailed description, and appended claims.

### BRIEF DESCRIPTION OF THE FIGURES

This specification contains at least one figure executed in color. Copies of this specification with color drawing(s) will be provided upon request and payment of the necessary fee. As those in the art will appreciate, the data and information represented in the attached figures is representative only and do not depict the full scope of the invention.

FIG. 1 shows a conventional needleless medical valve. The valve has double seal compression points that are at risk for microbial contamination due to their exposure to air.

FIGS. 2-9 depict several representative single-use sanitizing articles according to the invention.

FIG. 10 shows four color photographs of experimental results detailed in Example 3, below.

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

This invention concerns patentable single-use sanitizing articles that can be used to effectively and efficiently clean, disinfect, and preferably sterilize, exposed surfaces of medical line connectors, particularly needleless medical valves, as these surfaces are at risk for contamination with infectious agents such as bacteria, fungi, and viruses. “Single-use” (or “single purpose”) refers to an article or device suitable for one use or purpose only, as distinguished from “dual” or “multiple” use or purpose devices. Thus, in the context of the invention, a “single-use” sanitizing article or device is one that is useful for sanitizing, for example, a needleless medical valve or, at least with respect to some embodiments, a region of skin of a subject. After such use, the device is no longer suitable for any further use or purpose and is to be discarded. In contrast, a dual-use device would include one suitable for both sanitizing a medical fitting and then serving as a cap to minimize exposure of the valve to infectious agents when the valve is not being used to provide access to the patient's vasculature.

In general, the single-use sanitizing articles of the invention each comprise a sanitizing element disposed in a shell such that the sanitizing element can be maintained in a clean, preferably sterile, condition until it is used to sanitize (i.e., clean, disinfect, or sterilize) a medical line connector, such as a needleless medical valve. Herein, a sanitizing element comprises a sanitizing reagent dispersed in a substrate. In some embodiments, the sanitizing reagent is dispersed in or otherwise combined with the substrate during the process used to manufacture the sanitizing element, while in other embodiments, the device is configured such that the sanitizing reagent is released for dispersion into the substrate post-manufacture, but prior to the time the device is brought into contact with the needleless valve to be sanitized.

In accordance with the invention, a sanitizing reagent comprises an active ingredient capable of sanitizing a surface of a needleless medical valve. Any active ingredient that can be used effectively to rapidly sanitize a medical fitting or medical line connector (e.g., a needleless medical valve) can be adapted for use in practicing the invention, and are generally classified as antibacterial and antifungal agents, antiseptic or antimicrobial agents, wide spectrum disinfectants, and/or parasiticides, as well as combinations of such reagents. Particularly preferred are biocompatible active ingredients and sanitizing reagents, as the devices of the invention are intended for human and/or veterinary use, including alcohols, antibiotics, oxidizing agents, and metal salts. Representative examples of such active ingredients include bleach, chlorhexidine, ethanol, isopropyl alcohol, hydrogen peroxide, sodium hydroxide, and an iodophor dissolved or otherwise dispersed in a suitable solution, suspension, or emulsion. Other active ingredients having suitable sanitizing effects can also be used. These include alcohols (e.g., ethanol, benzyl alcohol, isopropyl alcohol, phenoxyethanol, phenethyl alcohol, etc.); antibiotics (e.g., aminoglycosides, such as amikacin, apramycin, gentamicin, kanamycin, neomycin, netilmicin, paromomycin, rhodostreptomycin, streptomycin, and tobramycin; bacitracin; chloramphenicol; erythromycin; minocycline/rifampin; tetracycline; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; etc.), quaternary ammonium chlorides; quaternary ammonium carbonates; benzalkonium chloride; chlorinated phenols; fatty acid monoesters of glycerin and propylene glycol; iodine; iodine containing compounds, such as 3-iodo-2-propynyl butyl carbamate (IPBC); iodophors, such as povidone-iodine (Betadine 100%, which contains providine iodine as the active ingredient); hydantoins, such as dimethylhydantoin and halogenated hydantoins; isothiazolinones; parabens, such as methylparaben, ethylparaben, and propylparaben; chloroxylenol; chlorhexidine and its salts; chlorhexidine/silver-sulfadiazine; chlorhexidine acetate; chlorhexidine gluconate (e.g., Hibiclens); chlorhexidine hydrochloride; chlorhexidine sulfate; benzoic acid and salts thereof; benzalkonium chloride; benzethonium chloride; methylbenzethonium chloride; chlorobutanol; sorbic acid and salts thereof; imidazole antifungals (e.g., miconazole); butoconazole nitrate; mafenide acetate; nitrofurazone; nitromersol; triclocarban; phenylmercuric nitrate or acetate 0.002%); chlorocresol; chlorbutol; clindamycin; CAE (Ajinomoto Co., Inc., containing DL-pyrrolidone carboxylic acid salt of L-cocoyl arginine ethyl ester); cetylpyridinium chloride (CPC) at 0.2%, 0.02%, and 0.002% concentrations; 9.8% isopropyl alcohol; 1% ZnEDTA; mupirocin; and polymyxin (polymyxin b sulfate-bacitracin). Additionally, other useful compounds and compositions include Miconazole, Econazole, Ketoconazole, Oxiconizole, Haloprogin, Clotrimazole, butenafine HCl, Naftifine, Rifampicin, Terbinafine, Ciclopirox, Tolnaftate, Lindane, Lamisil, Fluconazole, Amphotericin B, Ciprofloxecin, Octenidine, Triclosan (2,4,4′-trichloro-2′-hydroxydiphenyl ether), Microban (5-chloro-2-phenol (2,4dichlorophenoxy). Useful metals include silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine.

The particular active ingredient(s) selected as a sanitizing reagent for a given application will be compatible with the sanitizing element substrate and material(s) used to form the shell of the particular device. In some embodiments, the sanitizing reagent is dispersed in the substrate after the substrate is formed, for example, by saturating or supersaturating a substrate with the sanitizing reagent before or after it is coated or integrated with a pre-fabricated housing. In other embodiments, it is dispersed during the process used to manufacture the substrate. As will be appreciated, the materials used to prepare the sanitizing reagent should be compatible with the constituent or constituents that comprise the substrate such that the substrate does appreciably degrade or otherwise suffer loss of structural integrity prior to being used to sanitize a medical valve or region of a patient's skin. Similarly, the sanitizing reagent should be biocompatible, such that it will not harm a patient's skin the event of contact or should some amount of the sanitizing reagent inadvertently be admitted into the fluid carrying portion of a needleless medical valve, as well as with materials used to form needleless medical valves.

In preferred embodiments, the substrate used to form a sanitizing element is any suitable absorbent, pliable, fibrous or porous material, or combination of materials, than can be wetted and/or impregnated with a sanitizing reagent. Such materials include those that are synthetic or naturally occurring, and they may be of homogeneous or heterogeneous composition. Preferred synthetic materials include fibrous, foam, and gel compositions, particularly those having directionally oriented natural or synthetic fibers, or combinations thereof. Preferred naturally occurring materials useful as substrates include fibrous naturally occurring materials, including plant-derived materials such as cotton and paper products, as well as animal-based fiber products such as wool. Other preferred natural materials are sponges.

As will be appreciated, in order to achieve the desired sanitizing effect, a sanitizing element, or the component part(s) thereof designed to contact a medical fitting such as a needleless medical valve, preferably are made of a material (or combination of materials) that allow the sanitizing element to thoroughly sanitize surfaces of medical fittings such as needleless valves, particularly those surfaces that are exposed to air, and thus are at risk for contamination with infectious agents, and are also intended to form part of the fluid flow path for fluids to be introduced into a patient, for example, IV solutions, medications, blood and blood products, etc. Preferably, the substrate material should be sufficiently compliant to allow a medical fitting, particularly that portion of a needleless medical valve that contains the fluid access port, to be associated with, and in preferred device configurations, inserted into an article according to the invention, yet conform to the shape of the valve to assure intimate contact to at least those exposed surfaces of the valve intended to come into contact with fluid. In addition, the substrate allows for the retention of a liquid sanitizing reagent, for example, in capillary spaces, in the void volume of sponges, etc. The substrate may also be formulated such that its surface is modified to include sanitizing reagents such as silver ions and/or other suitable materials.

A particularly preferred class of materials for substrate fabrication is directionally oriented fibrous materials. These include, without limitation, materials comprised of cellulose fibers, glass fibers, and polyester fibers, as well as materials comprised of combinations of two of more of these and/or other materials. A particularly preferred fibrous substrate material is that used to form Transorb XPE® reservoirs (Filtrona Fibertec, Richmond, Va.). Such bonded synthetic fibers use capillary action to precisely absorb, retain, transfer, and/or release liquids or vapor in desired amounts. A broad range of synthetic polymers can be used to form the fibers, and, if desired, they may be treated for functional purposes, for example, to contain a sanitizing reagent dispersed therein, to provide a vapor barrier or other coating over a portion of the product's surface, etc. The geometric shape of these materials can also be customized for particular applications, thereby permitting easy integration of the substrate into desired device forms. Furthermore, the materials can include chemicals to indicate a functional change in the substrate, for example, by using a color change to signal a change from a wet to a dry state. In this way, a color change in the substrate could be used to indicate that the substrate has dried out and should not be used, perhaps due to a leak in the article's storage container.

Other representative classes of materials suitable for use as substrates include gel-forming polymers such as agarose, agar, polyacrylamide, and other synthetic porous materials that can be formed into layers, sheets, columns, or other shapes compatible with practicing the invention. Representative gelatinous materials include hydrogels (i.e., cross-linked polymers that absorb and hold water), particularly those made from agarose, (2-hydroxyethyl)methacrylate and its derivatives, and synthetic carbohydrate acrylamides.

Still other classes of materials include porous polymer sponges. Such sponges can be formed from any suitable material, including polyethylene, polypropylene, polytetrafluoroethylene, polyvinylidine difluoride, polynitrile, and polystyrene. Many such porous polymer sponges are commercially available in a wide variety of shapes, pore density and size, etc. Additionally, polymer sponges can be made by polymerizing appropriate monomers according to conventional foam forming techniques. In general, sponges have an open pore structure to allow movement of a solvent such as a liquid sanitizing reagent. The sponge surface should include open pores to provide entry of liquid sanitizing reagents (e.g., alcohol, iodine-containing solutions, etc.), and, as with other materials used to form substrates, the particular substrate material chosen is preferably inert, i.e., not reactive with components of the sanitizing reagent, the shell of the article or its container, or the materials used to produce medical fittings such as needleless medical valves.

Surgical foams are another preferred class of substrate materials. The materials can be natural or synthetic, as desired. Suitable foams include rubber latex, polyurethane, polyethylene and vinyl foams. Preferably, such foams are made from any suitable biocompatible polymer, for example, polyvinyl alcohol (PVA) or polyurethane. One preferred foam material is Microbisan™, a hydrophilic polyurethane foam that is impregnated with silver ions (Lendell Manufacturing, St. Charles, Mich.). Preferably, such foams are highly absorbent and thus suitable for use with liquid sanitizing reagents. In other embodiments, the material used to form the foam is well-suited for dispersion of a dry sanitizing reagent, such as silver ions. Again, it is preferred that foam materials, if used to as a substrate, be inert. Also, they are preferably sufficiently flexible to conform to the variety of different shapes and surface configurations (e.g., double seal fluid access points, luer threads, etc.) encountered in the field given the multitude of medical valve shapes, sizes, and configurations. In this way sufficient contact between the sanitizing surface(s) of the sanitizing element and the surface(s) of the medical valve to be cleansed can be ensured. Another advantage of some synthetic foams (as well as certain other polymeric materials from which substrates may be formed) is that they can easily be injected in a desired volume into a shell or housing during manufacture, after which they expand to assume the desired substrate size, density, porosity, etc.

Preferred natural materials include those derived from cotton and naturally occurring sponges. As those in the art appreciate, processed cotton fibers are composed almost entirely of the natural polymer cellulose. In such fibers, 20-30 layers of cellulose are coiled into a series of spring configurations, which makes the fibers absorbent and gives them a high degree of durability and strength. For example, woven cotton sheets, as are often used in the manufacture of sterile cleansing pads that are then saturated with a 70% isopropyl alcohol (IPA) solution, can be used as substrates. Any suitable configuration may be used. For example, a woven cotton sheet can be rolled to form a tube that can then be cut into small cylinders, before of after dispersing a suitable sanitizing reagent therein. In some embodiments of the invention, such cylinders can be used as substrates in the manufacture sanitizing elements that are then integrated with suitable shells or housings. Other fibers, be they naturally occurring, synthetic, or combinations of natural and synthetic materials, having similar properties can also readily be adapted for use as substrates to make sanitizing elements.

The sanitizing element of any substrate includes a sanitizing region capable of engaging an accessible surface of a valve stem of a needleless medical valve so as to expose the accessible surface, and any infectious agents residing thereon, to the sanitizing reagent. In many embodiments, the sanitizing region is the exposed, accessible surface (i.e., a sanitizing surface) of the sanitizing element designed to contact the surface to be sanitized, and the rest of the sanitizing element is inaccessible due to the shell or housing.

In some embodiments, an abrasive layer may be disposed on or comprises the upper surface of the substrate, such that the upper surface, or face, of the abrasive layer comes to for the sanitizing region of the sanitizing element. An abrasive layer typically is comprised of a natural or synthetic material, or combination of materials, that provide it with a greater abrasive or scrubbing capacity than material used to form the substrate, thereby enabling the abrasive layer to provide greater capacity to assist in the mechanical disruption or removal of biofilms (as, for example, may be formed by infectious agents contaminating the exposed surface(s) of needleless medical valves in a PIVC or CVC connected to a patient in a hospital or other healthcare setting) or other unwanted materials. It will also be understood that an “abrasive layer” can be formed in the upper portion of the substrate that includes the sanitizing region by a suitable treatment, such as heating, chemical treatment, and the like.

As already described, in some embodiments, the sanitizing element comprises a single layer, whereas in others, it comprises a plurality of layers. In multi-layer devices, the substrate used to form each layer can be of the same or different material, and may or may not contain a sanitizing reagent. Additionally, in some embodiments of multi-layer devices, one or more of the layers may be physically separated from the other layer(s) it contacts by an impermeable, semi-permeable, or permeable barrier.

For sanitizing elements that comprise multi-layered substrates, at least one of the layers contains a sanitizing reagent. In some such embodiments, each layer contains the same or a different sanitizing reagent. Here, a “different sanitizing reagent” means that each reagent contains either a different active ingredient(s), or the same active ingredient(s) in a different formulation or concentration. When different active ingredients are used, they are preferably compatible, such that one does not inactivate or otherwise degrade the sanitizing activity of the other active ingredient(s), nor should it materially degrade or chemically alter any substrate used to form a substrate layer or any material used to manufacture a medical fitting that can be sanitized by the device of the invention.

In embodiments wherein the sanitizing element is comprised of two or more layers, the substrate portion of each layer can be formed from a material that is the same as or different from the material used to form the substrate of one or more of the other layers, and each layer may contain the same, different, or even no, sanitizing reagent (although at least one layer will have a sanitizing reagent dispersed therein prior to engaging the surface of the needleless valve to be sanitized). Also, even when substrates for different layers are formed from the same material, they may be configured differently. For example, in a particularly preferred embodiment that employs a sanitizing element having two layers, where the substrate for each layer is formed from the same type of synthetic absorbent material having directionally fibers, the orientation of the fibers in one layer can differ from the fiber orientation in the other layer.

In any single-use sanitizing article according to the invention, the sanitizing element is encapsulated, enclosed, or housed in a suitable shell, housing, or other container or coating such that at least a portion of the sanitizing element, preferably its sanitizing region, is exposed for contact with a surface to be sanitized, for example, an accessible surface of a valve stem of a needleless valve of a medical fitting. Thus, in some embodiments, a sanitizing element is disposed in a pre-fabricated shell or housing, either during the manufacturing process or even in the field, where a sanitizing element is inserted or otherwise associated with a suitable shell, housing, or other container designed to accept a particular sanitizing element. In other embodiments, a sanitizing element is coated with one or more suitable materials. In yet other embodiments, a sanitizing element is loosely or securely packaged in a pouch (e.g., a foil pouch) or other container that is then sealed. A will be appreciated, a sanitizing element, particularly those that include a shell or housing, can also be adapted to be engaged by a gripping element of a machine designed to impart a twisting, rotating, and/or plunging action on a sanitizing article while it engages a medical fitting, such as a needleless medical valve to be sanitized prior to connection to a reservoir (e.g., an IV bag or syringe) for delivery of a solution to a patient.

Turning to embodiments wherein the shell is pre-fabricated, the shell can be produced using any suitable process (e.g., casting, extrusion, molding, and vacuum-forming) using any suitable material, or combination of materials, although materials amenable to various molding or forming processes are preferred. Representative materials include any suitable plastic or polymer, particularly medical grade plastics and urethanes. Preferred processes injection molding and vacuum-forming processes designed for use with thermoplastics.

A thermoplastic is a material that is plastic or deformable, melts to a liquid when heated and freezes to a brittle, glassy state when cooled sufficiently. Most thermoplastics are high molecular weight polymers whose chains associate through weak van der Waals forces (polyethylene); stronger dipole-dipole interactions and hydrogen bonding (nylon); or even stacking of aromatic rings (polystyrene). Many thermoplastic materials are addition polymers. These include vinyl chain-growth polymers such as polyethylene and polypropylene. Other thermoplastic polymers include acrylonitrile butadiene styrene, polyacrylates, polyacrylonitrile, polycarbonate, polyamides (including naturally and synthetic polyamide materials, e.g., nylons, aramids, etc.), polyester, polystyrene, polysulfone, polyvinyl chloride, cellulose acetate, ethylene-vinyl acetate (EVA), and fluoroplastics (including polytetrafluoroethylenes).

Thermoplastic polymers differ from thermosetting polymers in that the former can, unlike the latter, be remelted and remolded. Thermosetting plastics (thermosets) can also be used to make shells, and are polymer materials that are formed into desired shapes by curing, generally by heating, irradiation, or chemical reactions, to a stronger form that cannot be melted and re-shaped after curing. They are usually liquid or malleable prior to curing, and designed to be molded into their final form, or used as adhesives. Curing transforms the resin into a plastic or rubber by cross-linking of chemically active sites in the polymers, linking them into a rigid, solid three-dimensional structure. Thermosets are generally stronger than thermoplastics due to chemical cross-linking between polymer chains. Thermosets include vulcanized rubber, bakelite (a phenol formaldehyde resin), melamine resin, polyester resin (used in glass-reinforced plastics/fiberglass), and epoxy resin (used as an adhesive and in fiber-reinforced plastics).

Thermoplastic and thermoset materials can be shaped using any suitable process, including reactive Injection molding, extrusion molding, compression molding, blow molding, and spin casting. If necessary, the resulting parts may be machined or otherwise treated, for example, with a coating, after manufacture.

Other materials suitable for forming pre-fabricated shells or housings are thermoplastic elastomers. These materials are a class of copolymers or a physical mix of polymers (usually a plastic and a rubber) having both thermoplastic and elastomeric properties. While most elastomers are thermosets, thermoplastics are in contrast relatively easy to use in manufacturing, for example, by injection molding. Thermoplastic elastomers have features typical of rubbery materials and plastic materials. For example, they are elastic; however, unlike thermoplastics, they can not be remelted and remolded.

In addition to pre-fabricated shells or housings, sanitizing elements may instead be coated with any suitable material that can maintain sterility and prevent the sanitizing reagent from dissipating into the surrounding environment. Coatings including polymers such as plastics, rubbers, and other elastomeric materials that can be applied to the sanitizing element before or after the sanitizing reagent has been dispersed therein. A particular coating will be applied using a suitable process. Coating processes include dipping, spraying, and deposition. The process and coating material selected should not adversely effect the chemical activity of the active ingredient of the sanitizing reagent, and should be compatible in general with the material(s) of the substrate and be suited for use with devices and compositions intended for medical and/or veterinary use.

With regard to preferred embodiments wherein a sanitizing element is disposed within a grippable housing or shell, the housing typically contains an open cavity that defines a cleaning port adapted to receive a sanitizing element and engage an access point of a medical fitting, e.g., a catheter hub or similar article. Such a cavity is typically defined by an opening that allows a portion of the sanitizing element to be brought into contact with a medical line connector access point, a bottom disposed opposite the opening and upon which the sanitizing element is positioned, and at least one wall, the upper portion of which defines the opening and a lower portion of which adjoins the bottom. The cavity may be of any suitable size and shape, with the understanding that the particular configuration (i.e., size and shape) of the cavity preferably takes into account the configuration of the access point, e.g., catheter hub of a needleless valve, with which the sanitizing unit is designed to be engaged.

In preferred embodiments, the bottom of the cavity comprises a seat against which the sanitizing element is disposed. In some embodiments, such a seat comprises a substantially planar surface, whereas in others, the seat may comprise two or more portions positioned differently in relation to each other. For example, in some particularly preferred embodiments, a circular seat will comprise a substantially planar outer ring portion and an inner portion that protrudes above the outer ring portion when viewed from the side. The protruding, or raised, inner portion can have any desired shape, and can even be configured to contain a flange element elevated above the plane defining the upper surface of the seat's outer ring portion that can engage the sanitizing element and help to retain it.

As already described, the cavity of a cleaning unit can be of any suitable configuration. Cylindrical bore shapes of any desired width and depth are particularly preferred. Indeed, in some of these embodiments, the cylindrical bore can be configured to mate with a threaded portion, particularly when the access point to be cleaned is a threaded catheter hub (e.g., as used for intravenous lines, central venous lines). Examples of such threaded hubs include those that employ a luer lock connector. In other preferred embodiments, the cavity does not contain complementary surface features on the wall(s) of the bore designed to specifically mate with a threaded catheter hub, but is wide enough to so that the at the sanitizing element can be brought into contact with the threaded portion of the catheter hub when the sanitizing unit is brought into contact with the needleless valve.

Preferably, the outer surface of a shell has a non-slip surface, i.e., one having a high coefficient of friction so that when the sanitizing article is held in a user's hand and positioned to sanitize a medical fitting, it can be manipulated, for example, using a twisting or rotating motion, with minimal or no slippage in the user's bare or gloved hand. Examples of such surfaces include those having ridges, valleys, dimples, bumps, or other features designed to enhance friction, as well as combinations of two or more of such features. Such features can be introduced into the housing surface as part of the manufacturing process, and if desired in a particular application, materials having high grip levels can also be used to produce shells. Alternatively, a non-slip coating can be applied to at least the grippable portion of a housing.

In many preferred embodiments, a single-use sanitizing article of the invention also includes a seal secured to the housing, coating, or sanitizing element so as to cover the sanitizing region of the sanitizing element. Sealing can prevent tampering, mass transfer, and long-term stability. A suitable seal can be formed from any suitable material and can be attached to the shell using any suitable process. Preferably, the seal is formed from an impermeable material so as to prevent mass transfer (e.g., gas exchange, evaporation of a liquid sanitizing reagent from the sanitizing element, etc.) between the exterior environment and the interior of the sanitizing article. Suitable seal materials include foils and plastics. Depending on the seal material chosen, it is attached to the shell or housing a suitable process. For example, the seal may be adhered to the shell using an adhesive or other bonding agent that is biocompatible and also compatible with the materials used to form the sanitizing element and the shell or coating of the article.

One preferred sealing method is heat-sealing, preferably induction sealing. Induction sealing is a non-contact method of heating a metallic disk to hermetically seal the top of plastic or glass containers. The sealing process takes place after the sanitizing element has been placed, for example, into the cavity of a suitable plastic shell or housing. In such a method, the foil seal comprises a thin conductive metallic foil (e.g., aluminum foil) having a polymer film laminated to one surface of the foil. The seal is positioned over the opening in the housing. Once positioned, the seal is pressed down onto the lip of shell by the sealing head, the induction cycle is activated, and the seal is bonded to the shell. The induction cycle typically involves passing the seal and shell assembly under a sealing head having an induction coil, which emits a varying electromagnetic field. As the assembly passes under sealing head the conductive foil is heated. In a matter of seconds this heating causes the polymer film of the seal to heat and flow onto the lip of the shell. When cooled, the polymer creates a bond with the shell, resulting in a hermetically sealed assembly. Neither the shell nor the sanitizing element is affected. Such processes can be performed using a hand held unit or, for large-scale production, using an automated production line. In production line formats, the foil is typically provided in a reel, and an automated system is used to die cut and position individual foil seals with sanitizing articles to be sealed. In any event, the particular sealing conditions and equipment used will depend on such factors as the number of units to be manufactured, the particular configuration of the shell, the chemical compositions of the shell and sealing material, and the components of the sanitizing element. Conduction sealing another, albeit less preferred, heat sealing method that can also be used.

A seal can also be welded to the shell. An example of such a process is ultrasonic welding, whereby high-frequency ultrasonic acoustic vibrations are used to weld objects together, usually plastics, particularly molded thermoplastics, and especially for joining dissimilar materials.

The type of seal used will determine how it is to be removed, if at all. For example, in some embodiments, the seal is designed to be separated from the shell (or sanitizing element, if no shell is employed in the particular device), for example, by pealing, by a health care worker immediately prior to use in order to expose the sanitizing element prior to contacting it with medical fitting (e.g., a needleless medical valve) to be sanitized. In other embodiments, the seal may contain perforations or be scored or otherwise pre-fatigued so that the seal can easily be punctured in order to gain access to the sanitizing element disposed in the shell or housing, for example, by pressing a sanitizing element according to the invention that further comprises a puncturable seal against a needleless medical valve to be sanitized.

In other embodiments, the sanitizing element does not require a seal because it is packaged in a suitable container. For example, a sanitizing element can be packaged into a foil pouch or sleeve, which is then sealed and preferably then sterilized. In the context of the invention, such a pouch or sleeve may also be referred to as a shell or housing. In an example of such a process, a tubular sanitizing element is placed on a foil strip, which is then tightly wrapped around the length of sanitizing element and then sealed, for example, by heat-sealing. The foil is also sealed at either end of sanitizing element, with one or more notches, perforations, or the like cut or stamped into the sleeve to facilitate opening the sealed foil pouch also preferably being introduced at one or both ends of the package during the manufacturing process.

In some embodiments, the sanitizing article will include or otherwise be packaged with a drying element, i.e., an absorbent material designed to absorb residual sanitizing reagent from the surface(s) of the medical fitting contacted with a sanitizing article. In preferred embodiments, a drying elements is integrated into an article of the invention.

In general, the sanitizing articles of the invention are provided to users in a sealed, sterile manner. Typically this involves securing a seal to the shell to cover the access port, thereby enclosing the sanitizing element. After sealing, a sanitizing article is preferably packaged into a suitable container, for example, a foil pouch, for storage and transport. Of course, in embodiments wherein the shell is itself a foil pouch or sleeve, additional packaging of individual sanitizing articles is not necessary. If desired, labeling information, logos, artwork, manufacturing and regulatory data (e.g., lot number, expiration or “use by” dates, etc.) may also be printed or otherwise applied to individual sanitizing articles. In addition, information such as a bar code (to allow use of the device to tracked) may also be included on individual sanitizing articles. In particularly preferred embodiments, packaged sanitizing articles are sterilized using a suitable process, such as irradiation. As will be appreciated, sanitizing articles may be packaged individually or in groups of two or more units as kits, which can further include instructions for use of the sanitizing article(s).

In a particularly preferred practice, the sanitizing articles are sterilized as part of the manufacturing process. Here, “sterilization” refers to any process that effectively kills or eliminates transmissible agents, e.g., bacteria, viruses, fungi, prions, spores, etc. that may be present in any component of a device according to the invention. In preferred embodiments, sterilization can be achieved by heating, chemical treatment, irradiation, and other processes. Indeed, any sterilization process compatible with the materials used to make the sanitizing element can be employed. A particularly preferred sterilization process is an irradiation process. Such processes include irradiation with x-rays, gamma rays, or subatomic particles (e.g., an electron beam). In general, when a sterilization process is used in the context of the invention, the process is employed on a sanitizing article after it has been sealed and/or packaged.

The invention also concerns methods of using the instant single-use sanitizing articles. Such methods include using the articles to sanitize medical fittings such as needleless medical valves. To perform such methods, the sanitizing region of a single-use sanitizing article is contacted with the surface of the medical fitting to be sanitized, typically just before it is to be connected to a fluid-containing medical reservoir (e.g., an IV bag, syringe, etc.) that contains a solution to be delivered to a patient. In preferred practice, once in contact with the medical fitting, the article is moved in relation to the fitting, for example, by rotation or twisting. Such contact and sanitizing action can be for any desired period, with periods of about one second to about ten to twenty seconds being particularly preferred. After contact, the article is removed from the medical fitting, after which, for example, a fluid-containing medical reservoir is connected to the fitting. In preferred embodiments where the sanitizing reagent is a solution, the surface(s) of the fitting contacted with the sanitizing element are dried, either by evaporation or through contact with a sterile, dry, highly absorbent material prior to connection with the fitting. It will be appreciated that the articles of the invention can be used manually. Of course, one or more of single-use sanitizing articles can also be inserted (individually or in magazines) into a hand-held machine that, when properly positioned in relation to a needleless medical valve, allows the exposed surfaces of the valve to be sanitized upon actuation of the machine.

### REPRESENTATIVE EMBODIMENTS

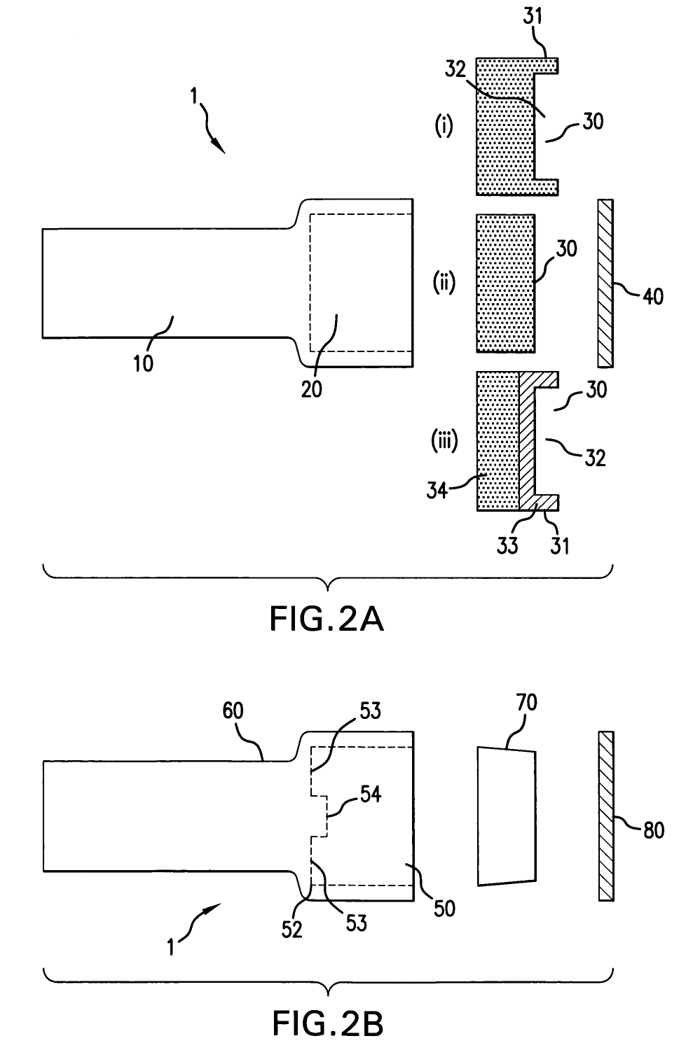

The following descriptions concern several representative embodiments of the invention, which are described in FIGS. 2-7.

FIG. 2 depicts two preferred embodiments of a single-use sanitizing article according to the invention. Panel A shows and exploded view wherein the sanitizing article (1) comprises a grippable housing (10) that contains a cavity (20) adapted to receive a sanitizing element (30) and mate with an access point of a medical fitting (not shown), e.g., a catheter hub or similar article. Three different sanitizing element configurations are shown (i, ii, and iii), any one of which can be disposed in the cavity. Sanitizing element configuration (i) shows a cross section of a sanitizing element wherein the substrate is made from one material, but wherein the portion (31) of the substrate proximate to the cavity opening has a void (32) to facilitate maximum surface engagement with the connector of a medical fitting to be cleaned. In configuration (ii), the substrate does not include a void. Configuration (iii) includes a void (32) in the substrate proximate to the cavity opening, as does the substrate in configuration (i). In configuration (iii), however, the substrate (30) comprises two layers (33 and 34). Preferably, the sanitizing article also includes a seal (40) secured to the housing so as to cover the opening at one end the cavity. Panel B of FIG. 2 shows a sanitizing article (1) similar to that depicted in Panel A; however, in panel B, the cavity (50) in the housing (60) comprises a seat (52) having two parts, a substantially planar outer portion (53) and a raised inner portion (54). The sanitizing element (70) may be adapted to conform to the seat's shape, if desired, but need not be. By using a seat having two parts, the center portion of the sanitizing element should compress less upon contact with the valve surface of a needleless medical valve, ensuring good contact.

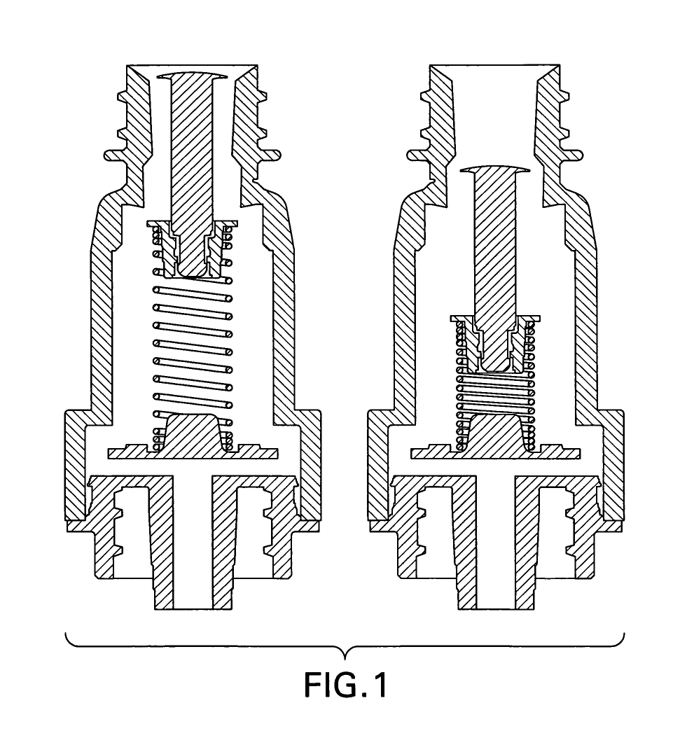

FIG. 3 shows a cutaway view of a single-use sanitizing device (80) that includes a sanitizing element (90) that contains a sanitizing reagent. In this embodiment, all of the outer surface the sanitizing element (90), except for the sanitizing region (91), is coated with a coating (100). Preferably, such a coating provides a grippable, non-slip surface over at least a portion of the coating's outer surface. The coating also limits, and preferably prevents, mass transfer between the inside of the article and the outside environment until such time as the article is ready to be used to sanitize surface of a medical fitting. A seal (110) covers the uncoated end of the article, and upon removal makes the sanitizing region (91) accessible so that it can be brought into contact with, for example, the valve stem of a needleless medical valve as depicted in FIG. 1.

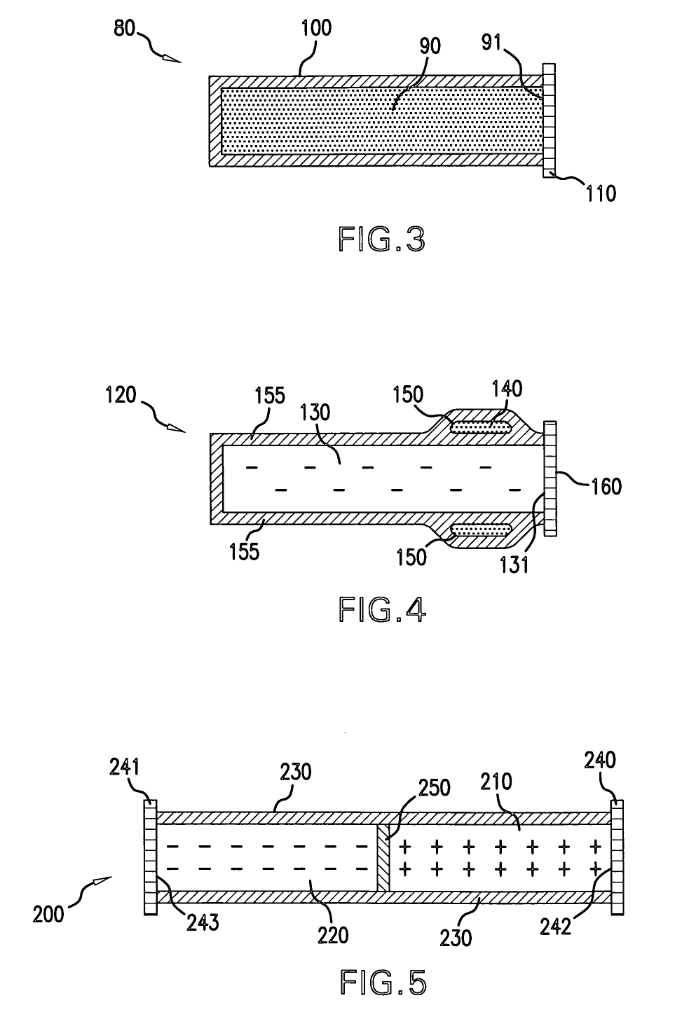

FIG. 4 shows a cutaway view of a single-use sanitizing device (120) that includes a sanitizing element (130) in which a sanitizing reagent (140) has not been dispersed or activated. Instead, the sanitizing reagent, a portion thereof, is stored in a reservoir (150) within the coating (155), such as a crushable bladder, that can be ruptured by a user just prior to engaging the sanitizing article with the medical fitting to be sanitized; the reservoir is not ruptured by merely engaging the sanitizing article and medical fitting. In this way, the sanitizing reagent (140) can become dispersed in the sanitizing element substrate, or, if dispersed in the substrate in an inactive form to become active, so that the sanitizing reagent is present in the sanitizing region (131) and ready for immediate use upon removal of the seal (160).

FIG. 5 shows a cutaway view of a single-use sanitizing device (200) that includes both a sanitizing element (210) and a drying element (220). Both the sanitizing element (210) and a drying element (220) are coated with a coating (230) and sealed with seals (240 and 241) to cover and protect the sanitizing surface (242) of the sanitizing element (210) and the drying surface (243) a drying element (220). The sanitizing element (210) and drying element (220) are separated by a barrier (250). The barrier is preferably an impermeable material or coating that prevents mass transfer, particularly transfer of the sanitizing reagent, between the sanitizing element (210) and the drying element (220). The sanitizing element (210) and drying element (220) can be made from the same or different material(s).

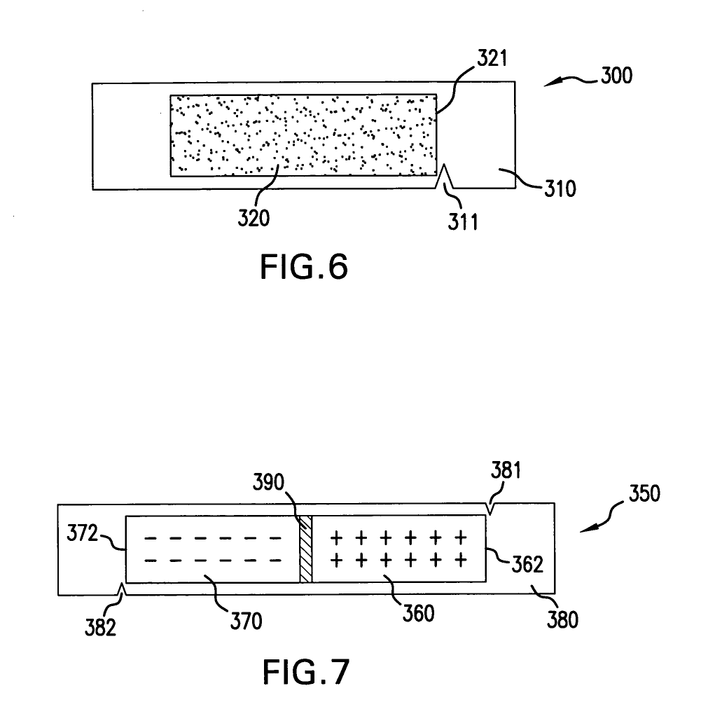

FIG. 6 shows a cutaway view of a single-use sanitizing device (300) sealed fixedly in a foil pouch (310) having a tear notch (311). The foil pouch is fixedly sealed around the sanitizing element (320) to prevent translation of the sanitizing element (320) within the foil pouch (310). In the embodiment shown, the sanitizing element (320) has an active sanitizing reagent dispersed therein; however, in other embodiments, the chamber within the pouch could also contain a breakable or crushable reservoir that a user could rupture just prior to opening the pouch by tearing at the tear notch (311). Opening the pouch exposes the sanitizing region (321) of the sanitizing element (320).

FIG. 7 shows a cutaway view of a single-use sanitizing device (350) that includes both a sanitizing element (360) and a drying element (370) sealed fixedly in a foil pouch (380) having tear notches (381 and 382). The foil pouch (380) is fixedly sealed around the sanitizing element (360) and drying element (370) to prevent their translation within the foil pouch (310). The foil pouch covers and protects the sanitizing surface (362) of the sanitizing element (360) and the drying surface (372) a drying element (370). The sanitizing element (360) and drying element (370) are separated by a barrier (390). The barrier is preferably an impermeable material or coating that prevents mass transfer, particularly transfer of the sanitizing reagent, between the sanitizing element (360) and the drying element (370). The sanitizing element (360) and drying element (370) can be made from the same or different material(s).

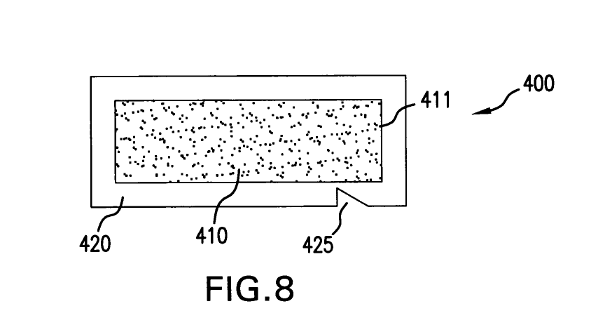

FIG. 8 shows a cutaway view of a single-use sanitizing device (400) that includes a sanitizing element (410) sealed fixedly in a foil sleeve (420) having a tear notch (425). The foil sleeve is fixedly sealed around the sanitizing element (410) to prevent translation of the sanitizing element (410) within the foil sleeve (420). In the embodiment shown, the sanitizing element (410) has an active sanitizing reagent dispersed therein. Opening the sleeve exposes the sanitizing region (411) of the sanitizing element (410). As those in the art will appreciate, an article such as depicted in FIG. 8 can be used either to sanitize a needleless medical valve or another surface, for example, a region of a patient's skin.

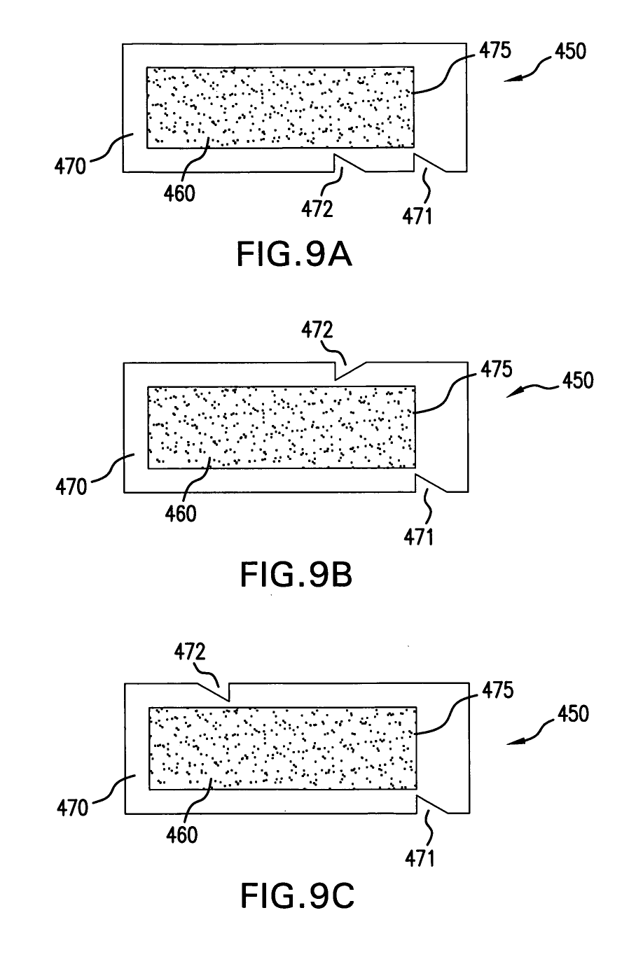

FIG. 9 shows three cutaway views, A, B, and C, of a single-use sanitizing device (450) similar to that shown in FIG. 8, the difference being that the device (450) in each view comprises a plurality of tear notches (471 and 472). As between views A, B, C, the difference is in the location of tear notches 471 and 472. As in the device shown in FIG. 8, the device depicted in FIG. 9 also includes a sanitizing element (460) sealed fixedly in a foil sleeve (470). The foil sleeve is again fixedly sealed around the sanitizing element (460) to prevent translation of the sanitizing element (460) within the foil sleeve (470). In the embodiment shown, the sanitizing element (460) has an active sanitizing reagent dispersed therein. Depending upon which tear notch is used to open the article, differing portions of the sanitizing element (460) are exposed. For example, when tear notch 471 is used to open the device, only a small portion, if any, of the sides of the sanitizing element (460) are exposed, in addition to the sanitizing region (475). On the other hand, if tear notch 472 is used to open the device (450), a portion of the sanitizing element (460) below and adjacent to the sanitizing region (475) is exposed. As will be appreciated, opening the device using either of the tear notches (471 or 472) allows the device to be used to sanitize a medical valve fitting (not shown). On the other hand, if a healthcare worker wishes to use the device (450) to sanitize a skin region, s/he would preferably use tear notch 472 to open the device, as that would expose more of the sanitizing element and reduce the risk the patient might be scratched or otherwise injured by the portion of the foil sleeve (470) remaining after the device is opened.

### EXAMPLES

The invention will be better understood by reference to the following Examples, which are intended to merely illustrate certain aspects and embodiments of the invention. The scope of the invention is not to be considered limited thereto.

### Example 1

### Analysis of Contaminated Needleless Valves Following Sanitizing Treatment

This example describes an assay for testing the effectiveness of sanitizing a needleless medical valve contaminated with a bacterial biofilm. This example also reports data demonstrating that sanitizing articles according to the invention are more effective at cleaning needleless medical valves than conventional valve-cleaning techniques.

The assay begins by inoculating a needleless medical valve with an aliquot of an inoculum containing a viable microorganism. Here, a 5 microliter (uL) aliquot from a log phase liquid culture of Geobacillus stearthermophilus was inoculated directly onto the surface of the access port of each of several Smartsite® needleless medical valves (B. Braun Medical Inc., Bethlehem, Pa.). In addition, a 10 uL aliquot from the same culture was also inoculated directly onto the luer threads of each of the Smartsite® valves. The valves were then left undisturbed for 30 min. at 35° C. For each of the different device classes tested with a device according to the invention or a conventional IPA-saturated pad, four or five contaminated valves were used. Two Smartsite® valves also contaminated with the same amount of the G. stearthermophilus inoculum served as positive controls. Two additional Smartsite® valves that had not been contaminated were used as negative, uncontaminated controls.

After 30 minutes, each of the test and control valves was sanitized as follows using either one of three different a sanitizing article configurations (configurations 1, 2, and 3) according to the invention or a conventional sterile cleansing pad saturated with a 70% isopropyl alcohol (IPA) (Webcol®, Kendall Co., Mansfield, Mass.). The sanitizing element of each of the devices of configuration 1 comprised a Filtrona® substrate saturated with 70% IPA. In the devices of configurations 2 and 3, the sanitizing elements were made from surgical foam that had been saturated with 70% IPA. The difference between configurations 2 and 3 was that in configuration 2, the sanitizing element was a continuous foam insert, whereas in configuration 3, the foam plug had been cored such that a cavity existed at one end of the sanitizing element to facilitate sanitizing of both a given Smartsite® valve's fluid access port and threaded luer portion.

In each case, the sanitizing device, be it an article according to the invention or a conventional IPA-saturated pad, was manually brought into contact with the previously inoculated surfaces of the access port and luer threads of an Smartsite® valve by gently pressing the device onto the valve. The device was then rotated back and forth several times in relation to the valve, after which the device was removed from contact with the valve and discarded. Each valve was allowed to air dry in a HEPA-filtered airflow for at least 30 seconds.

Following sanitizing treatment, under sterile conditions each of the valves under test was transferred to a separate 100 mL beaker containing a small magnetic stir bar and 20 mL of sterile saline solution (1×PBS, 137 mM NaCl, 10 mM sodium phosphate, 2.7 mM KCl, pH 7.4). Each beaker was then placed on a stir plate and the valve-plus-solution was stirred slowly for 2 min. Microorganisms were then collected from the solutions by filtering each solution through a separate 0.45 micron membrane filter. The filters were then placed on fresh TSA plates and incubated at 30° C.-35° C. for 48 hours. After the incubation, colonies were counted to determine the number of colony forming units (CFUs) in each filtrate. The plates for the two positive control valves had 179 and 187 colonies, respectively. The negative, uncontaminated control plates each had 0 colonies, as expected. Plates for each of the valves cleaned with conventional IPA-saturated pads averaged about 40 colonies, whereas plates overlaid with filters containing the filtrates from the Smartsite® valves that had been sanitized with a sanitizing article of configuration 1 had no colonies (0 CFU). For the devices of configuration 2, the average number of residual, post-treatment CFUs was 21, and for the devices of configuration 3, there were 18 CFUs remaining after treatment with a device according to the invention. Together, these results demonstrate that sanitizing articles according to the invention provide superior sanitizing action when used to clean needleless medical valves, as compared to the conventional widely used technique of swabbing the needleless valve with a 70% IPA wipe. Moreover, sanitizing a contaminated needleless medical valve with at least one of the invention's device configurations (configuration 1) completely eliminated the contaminating G. stearthermophilus microorganisms introduced onto the surfaces of the valve near or in the path fluids must traverse to enter the valve.

### Example 2

### Assay for Assessing Effectiveness of Sanitizing Contaminated Needleless Valves

This example describes an assay for testing the effectiveness of sanitizing a needleless medical valve contaminated with a bacterial biofilm. This assay is similar to that described in Example 1, the difference being that after the contaminated needleless medical valves are disinfected, they are individually placed in a sterile chamber (e.g., a plastic 90 mm Petri dish) and allowed to incubate at 30° C.-35° C. for 48 hours. The incubation period is intended to allow contaminating microorganisms that remain on the contaminated but sanitized surface(s) to recover before being collected onto a 0.45 micron filter and transferred to a plate containing nutrient agar for outgrowth and CFU enumeration.

### Example 3

### Visual Assay for the Assessing Effectiveness of Sanitizing Contaminated Needless Valves

This example describes an assay for testing the effectiveness of sanitizing a needleless medical valve contaminated with a microorganism engineered to fluoresce under ultraviolet light. This example also demonstrates that sanitizing articles according to the invention are more effective at cleansing needleless medical valves than conventional valve-cleaning techniques.

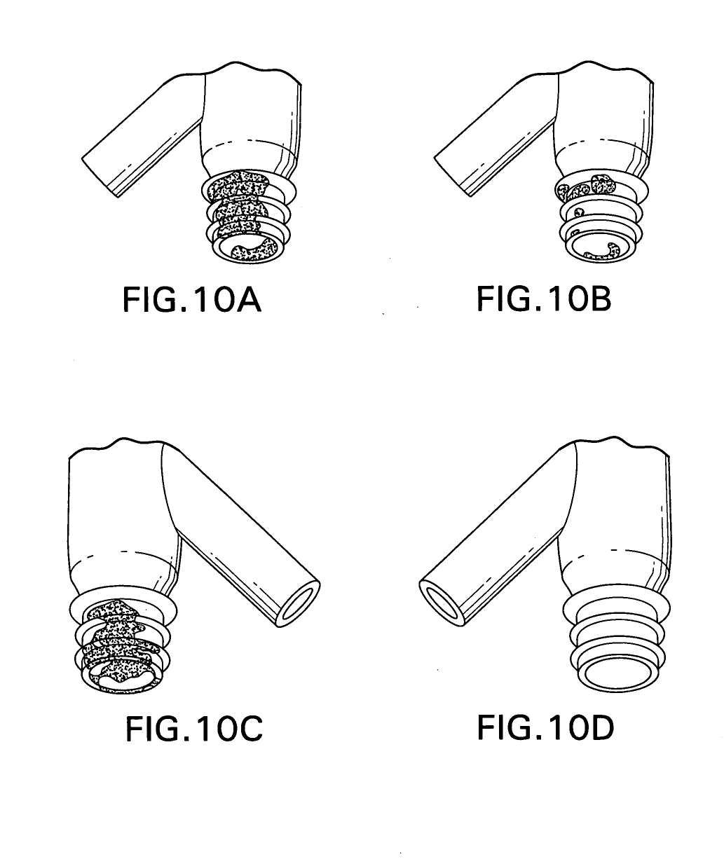

Here, the assay involved applying approximately 100 uL of Glo Germ™ (Glo Germ™ Co., Moab, Utah) to the surface to the access port and luer threads of each of 2 ULTRASITE® needleless medical valves (B. Braun Medical Inc., Bethlehem, Pa.). Post-inoculation, each valve was photographed under ultraviolet light (see FIG. 10, photos A and C). Each valve was then sanitized using either a sanitizing article of configuration 1 saturated with 70% IPA or a sterile, commercially available cleansing pad saturated with a 70% IPA. The valves were sanitized using the same procedure as in Example 1. After sanitizing, each valve was again photographed under ultraviolet light. FIG. 10, photo B, shows the results obtained using a commercially available cleansing pad saturated with a 70% IPA, while FIG. 10, photo D, shows the results of sanitizing using a sanitizing article according to the invention. As shown in FIG. 10, a commercially available cleansing pad saturated with a 70% IPA did not clean relevant valve surfaces (either the exposed surface of the valve's fluid access port or the luer threads) as thoroughly as did the sanitizing article of the invention.

All of the compositions, articles, and methods described and claimed herein can be made and executed without undue experimentation in light of the present disclosure. While the, articles and methods of this invention have been described in terms of preferred embodiments, it will be apparent to those of skill in the art that variations may be applied to the articles, methods, and compositions without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A patentable single-use article configured to sanitize a needleless valve of a medical fitting, comprising:
a. a sanitizing element comprising a substrate and a sanitizing reagent dispersed in the substrate prior to use, wherein the substrate has a sanitizing region capable of engaging an accessible surface of a valve stem of a needleless valve of a medical fitting, which needleless valve optionally comprises a threaded valve body adapted to engage a complementary threaded portion of a fluid delivery device; and
b. a shell disposed about the substrate and having an access port that allows the sanitizing region of the substrate to be brought into contact with and sanitize an accessible surface of a valve stem of a needleless valve of a medical fitting.

2. An article according to claim 1 further comprising a seal secured to the shell and covering the access port of the shell, wherein the article is optionally sterile.

3. An article according to claim 1, wherein the substrate of the sanitizing element comprises an absorbent material selected from the group consisting of a naturally occurring material and a synthetic material, wherein when the substrate is a naturally occurring material, it is optionally a natural sponge, and wherein when the substrate is a synthetic material, it is optionally selected from the group consisting of a fibrous composition, a foam, and a gel.

4. An article according to claim 1, wherein the sanitizing reagent is a liquid formulation, optionally an aqueous solution.

5. An article according to claim 1, wherein the sanitizing reagent comprises a sanitizing compound selected from the group consisting of an alcohol, chlorhexidine hydrogen peroxide, iodine, and silver ions.

6. An article according to claim 1, wherein the shell is applied to the sanitizing element, optionally as a coating, wherein the coating is optionally applied to the sanitizing element by a process selected from the group consisting of dipping, spraying, and deposition.

7. An article according to claim 1, wherein the shell comprises a prefabricated housing in which the sanitizing element is disposed, wherein the prefabricated housing optionally comprises a cavity, optionally comprising a cylindrical bore, defined by at least one wall and a bottom disposed opposite an opening that is the access port and which provides access to the cavity, wherein the cavity is configured to receive the sanitizing element, and wherein the cavity is further optionally configured to mate with an engaging element disposed on a valve body of a needleless medical valve, and wherein article is optionally sterile and further optionally comprises a seal secured to the housing and covering the access port of the cavity.

8. An article according to claim 7, wherein (i) the bottom of the cavity comprises a seat against which the sanitizing element is disposed, wherein the seat optionally comprises a substantially planar surface, optionally a substantially planar outer portion and an inner portion protruding above the outer portion, and/or (ii) at least a portion of an outer surface of the housing comprises a non-slip surface, wherein the non-slip surface optionally comprises of plurality of valleys and/or ridges disposed in the outer surface of the housing, and/or (iii) the housing is tapered along its length, and is optionally conical over at least a portion of its length.

9. A kit comprising an article according to claim 1 packaged in a single-use container, wherein the kit optionally further comprises instructions for use of the article.

10. A kit comprising a plurality of articles according to claim 1, wherein each article is separately packaged in a single-use container.

11. A patentable single-use article configured to sanitize a needleless valve of a medical fitting, comprising:
a. a sanitizing element comprising a substrate and a sanitizing reagent dispersed in the substrate prior to use, wherein the substrate has a sanitizing region capable of engaging an accessible surface of a valve stem of a needleless valve of a medical fitting;
b. a shell disposed about the substrate and having an access port that allows the sanitizing region of the substrate to be brought into contact with and sanitize an accessible surface of a valve stem of a needleless valve of a medical fitting; and
c. a seal secured to the shell and covering the access port of the shell,
wherein the article optionally is sterile.

12. A kit comprising an article according to claim 11 packaged in a single-use container, wherein the kit optionally further comprises instructions for use of the article.

13. A kit comprising a plurality of articles according to claim 11, wherein each article is separately packaged in a single-use container.

14. A patentable single-use article configured to sanitize a needleless valve of a medical fitting, comprising:
a. a sanitizing element comprising a substrate and a sanitizing reagent dispersed in the substrate prior to use, wherein the substrate has a sanitizing region capable of engaging an accessible surface of a valve stem of a needleless valve of a medical fitting;
b. a prefabricated housing in which the sanitizing element is disposed, wherein the prefabricated housing (i) comprises a cavity defined by at least one wall and a bottom disposed opposite an access port that provides access to the sanitizing element disposed in the cavity and (ii) is further configured to mate with an engaging element disposed on a valve body of a needleless valve of a medical fitting; and
c. a seal secured to the housing and covering the access port of the cavity,
wherein the article optionally is sterile.

15. A kit comprising an article according to claim 14 packaged in a single-use container, wherein the kit optionally further comprises instructions for use of the article.

16. A kit comprising a plurality of articles according to claim 14, wherein each article is separately packaged in a single-use container.

17. A patentable method of sanitizing an accessible surface of a valve stem of a needleless valve of a medical fitting, comprising contacting an accessible surface of a valve stem of a needleless valve of a medical fitting with a single-use sanitizing article according to claim 1 to sanitize the accessible surface, thereby sanitizing the accessible surface of the valve stem of the needleless valve of the medical fitting.

18. A patentable method of reducing infection risk in a patient connected to a venous catheter having at least one medical fitting having a needleless valve, comprising contacting an accessible surface of a valve stem of a needleless valve of a medical fitting of the venous catheter with a single-use sanitizing article according to claim 1 so as to sanitize the accessible surface of a valve stem of a needleless valve, thereby reducing infection risk in the patient.
